(19) United States
(12) Patent Application Publication
(10) Pub. No.: US 2007/0073243 A1
(43) Pub. Date: Mar. 29, 2007

(54) Fluid dosing device comprising gear transmission actuator

(76) Inventor: Stanley George Bonney, Ware (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/597,702

(22) PCT Filed: Feb. 4, 2005
(86) PCT No.: PCT/GB05/00399

§ 371 Date: Aug. 4, 2006

(30) Foreign Application Priority Data
Feb. 6, 2004 (GB) 0402698.5

(51) Int. Cl. A61M 5/178

(52) U.S. Cl. 604/186

(57) ABSTRACT

A delivery device for delivering a metered amount of substance on each actuation thereof that includes a delivery unit operable to deliver a metered amount of substance, wherein the delivering unit includes a metering cavity in fluid communication with a reservoir for storing substance, and a piston member reciprocatingly movably disposed in the metering cavity along a piston axis, the piston member being movable in a first direction to a first, primed position to draw substance into the metering cavity and thereby prime the delivery unit, and a second direction, opposite to the first direction, to a second, delivered position to meter and deliver a metered amount of substance from the metering cavity; and an actuation mechanism actuatable by a user to operate the delivery unit, the actuation mechanism that includes comprising an actuator member to which a user applies an actuation force.

### FIELD OF THE INVENTION

The present invention relates to a delivery device for delivering substance on each actuation thereof, and particularly, but not exclusively, a manually-actuatable delivery device for delivering a metered amount of substance on each actuation thereof.

### BACKGROUND OF THE INVENTION

Delivery devices which provide for the metering of substance are known in the art. As an example, in the medical field, the use of metered dose inhalers (MDIs) is well established. In an MDI, substance is contained under pressure in a canister having an open end closed by a valve mechanism. The valve mechanism has a valve body which defines a metering chamber of fixed volume and a valve stem which is movable between filling and discharging positions. In the filling position, the valve stem places the metering chamber in fluid communication with the content of the canister, but isolates the metering chamber from the external environment. In the discharge position, the valve stem places the metering chamber in fluid communication with the external environment, but isolates the metering chamber from the content of the canister. In this way, a metered volume of substance is sequentially transferred to the metering chamber and then discharged to the external environment for inhalation by a user.

The present invention aims to provide an improved delivery device for delivering a metered amount of substance on each actuation thereof, and in particular a manually-actuatable delivery device for delivering a metered amount of substance on each actuation thereof.

### SUMMARY OF THE INVENTION

In one aspect the present invention provides a delivery device for delivering a metered amount of substance on each actuation thereof, comprising: a delivery unit operable to deliver a metered amount of substance, the delivery unit including a piston member which, in a reciprocating movement, primes, meters and delivers a metered amount of substance; and an actuation mechanism actuatable by a user to operate the delivery unit, the actuation mechanism comprising an actuator member to which a user applies an actuation force in substantially a single direction, and being configured to cause the reciprocating movement of the piston member on application of the actuation force.

In one embodiment the delivery unit comprises a metering cavity in fluid communication with a reservoir for storing substance, and the piston member is reciprocatingly movably disposed in the metering cavity along a piston axis, the piston member being movable in a first direction to a first, primed position to draw substance into the metering cavity and thereby prime the delivery unit, and a second direction, opposite to the first direction, to a second, delivered position to meter and deliver a metered amount of substance from the metering cavity.

Preferably, the actuation mechanism further comprises a gear assembly which is operably coupled to the piston member and the actuator member such as to effect the reciprocating movement of the piston member on application of the actuation force to the actuator member.

More preferably, the gear assembly comprises a first, drive gear which is rotatable about a pivot and driven by actuation of the actuator member, and a second, driven gear which is rotatable about a pivot, driven by the drive gear and operably coupled to the piston member.

In one embodiment the actuation mechanism further comprises a drive member which operably couples the piston member and the driven gear.

Preferably, the drive member is pivotally mounted about a pivot such as to be reciprocatingly pivoted by the driven gear.

In one embodiment the drive member is configured such that the piston member is moved at a greater speed in the first direction during a delivery stroke in which substance is delivered from the metering cavity than in the second direction during a priming stroke over a length equal to the delivery stroke in which substance is drawn into the metering cavity.

Preferably, the pivot of the drive member is located in a position offset from an axis extending through the pivot of the driven gear and orthogonal to the piston axis.

Preferably, the driven gear includes a drive pin on a face thereof which engages the drive member.

In one embodiment the drive gear includes n lugs on a face thereof, the lugs being equi-angularly spaced and having an angular spacing of 360/n degrees, and the actuator member is configured to rotate the drive gear through 360/n degrees on each actuation thereof.

Preferably, the driven gear has a diameter of 1/n of that of the drive gear such that, for each actuation of the actuator member, the driven gear is rotated through 360 degrees.

In one embodiment the metering cavity includes a peripheral wall and the piston member includes a piston which is a sealing fit with the peripheral wall of the metering cavity, the piston having a pressure face for acting on the substance.

Preferably, the peripheral wall of the metering cavity includes a transfer port, the transfer port being located at a position between the positions of the pressure face of the piston when the piston member is in the primed and delivered positions, and providing for the transfer of substance from the metering cavity to the reservoir with movement of the piston member in the second direction until closed by the piston.

In one embodiment, prior to actuation of the actuator member, the piston member is located such as to close the transfer port.

Preferably, prior to actuation of the actuator member, the piston member is located such as to close the metering cavity from an external environment.

Preferably, the device further comprises: a storage unit including a reservoir for storing substance in fluid communication with the delivery unit.

Preferably, the device further comprises: an outlet unit in fluid communication with the delivery unit from which substance is delivered.

More preferably, the outlet unit comprises a spray nozzle.

In another aspect the present invention provides a delivery device for delivering a metered amount of substance on each actuation thereof, comprising: a delivery unit operable to deliver a metered amount of substance, wherein the delivery unit comprises a metering cavity in fluid communication with a reservoir for storing substance, and a piston member reciprocatingly movably disposed in the metering cavity along a piston axis, the piston member being movable in a first direction to a first, primed position to draw substance into the metering cavity and thereby prime the delivery unit, and a second direction, opposite to the first direction, to a second, delivered position to meter and deliver a metered amount of substance from the metering cavity; and an actuation mechanism actuatable by a user to operate the delivery unit, the actuation mechanism comprising an actuator member to which a user applies an actuation force, and being configured to effect the reciprocating movement of the piston member on application of the actuation force, with the piston member being moved at a greater speed in the first direction during a delivery stroke in which substance is delivered from the metering cavity than in the second direction during a priming stroke over a length equal to the delivery stroke in which substance is drawn into the metering cavity.

Preferably, the actuator member is configured such that a user applies the actuation force in substantially a single direction.

In one embodiment the actuation mechanism further comprises a gear assembly which is operably coupled to the piston member and the actuator member such as to effect the reciprocating movement of the piston member on application of the actuation force to the actuator member.

Preferably, the gear assembly comprises a first, drive gear which is rotatable about a pivot and driven by actuation of the actuator member, and a second, driven gear which is rotatable about a pivot, driven by the drive gear and operably coupled to the piston member.

In one embodiment the actuation mechanism further comprises a drive member which operably couples the piston member and the driven gear.

Preferably, the drive member is pivotally mounted about a pivot such as to be reciprocatingly pivoted by the driven gear.

In one embodiment the pivot of the drive member is located in a position offset from an axis extending through the pivot of the driven gear and orthogonal to the piston axis.

Preferably, the driven gear includes a drive pin on a face thereof which engages the drive member.

In one embodiment the drive gear includes n lugs on a face thereof, the lugs being equi-angularly spaced and having an angular spacing of 360/n degrees, and the actuator member is configured to rotate the drive gear through 360/n degrees on each actuation thereof.

Preferably, the driven gear has a diameter of 1/n of that of the drive gear such that, for each actuation of the actuator member, the driven gear is rotated through 360 degrees.

In one embodiment the metering cavity includes a peripheral wall and the piston member includes a piston which is a sealing fit with the peripheral wall of the metering cavity, the piston having a pressure face for acting on the substance.

Preferably, the peripheral wall of the metering cavity includes a transfer port, the transfer port being located at a position between the positions of the pressure face of the piston when the piston member is in the primed and delivered positions, and providing for the transfer of substance from the metering cavity to the reservoir with movement of the piston member in the second direction until closed by the piston.

In one embodiment, prior to actuation of the actuator member, the piston member is located such as to close the transfer port.

Preferably, prior to actuation of the actuator member, the piston member is located such as to close the metering cavity from an external environment.

Preferably, the device further comprises: a storage unit including a reservoir for storing substance in fluid communication with the delivery unit.

Preferably, the device further comprises: an outlet unit in fluid communication with the delivery unit from which substance is delivered.

More preferably, the outlet unit comprises a spray nozzle.

### BRIEF DESCRIPTION OF THE DRAWINGS

A preferred embodiment of the present invention will now be described hereinbelow by way of example only with reference to the accompanying drawings, in which:

FIG. 1 illustrates a part-sectional elevational view of a delivery device in accordance with a preferred embodiment of the present invention; and

FIGS. 2 to 11 illustrate part-sectional elevational views of the delivery device of FIG. 1 in various operative states in a single actuation operation.

### DETAILED DESCRIPTION OF THE PREFERRED EMBODIMENT

The delivery device which will now be described with reference to the accompanying Figures of drawings implements the basic principle of operation for dispensing a metered volume of a fluid disclosed in Applicant's co-pending International patent application Nos. PCT/EP03/08646 and PCT/EP03/08647, the entire contents of each of which are incorporated herein by reference.

The delivery device comprises a storage unit 3 for storing a fluid substance, in this embodiment a liquid containing a medicament in suspension or solution, which is to be delivered by the device, an outlet unit 5 from which substance is delivered, a delivery unit 7 which is in fluid communication with the storage unit 3 and the outlet unit 5 and operable to meter a predetermined volume of substance from the storage unit 3 to the outlet unit 5 on operation thereof, and a manually-actuatable actuation mechanism 9 which is coupled to the delivery unit 7 such as to provide for operation of the same on actuation of the actuation mechanism 9.

In this embodiment the storage unit 3 comprises a body 11 which includes a storage cavity 15, which storage cavity 15 has a peripheral wall 16 and a closed end 17 including a transfer port 18, and a transfer channel 19 which is fluidly connected to the transfer port 18 in the closed end 17 of the storage cavity 15, and a sealing member 21 which is slideably disposed in the storage cavity 15 such as to be a sealing fit with the peripheral wall 16 thereof and define a reservoir 23 containing substance. The sealing member 21, in being a free sliding fit with the peripheral wall 16 of the storage cavity 15, advances with each withdrawal of substance from the reservoir 23 so as to provide that the reservoir 23 contains only substance, and, more particularly, that substance is always present at the transfer port 18 in the closed end 17 of the storage cavity 15. The body 11 also includes a guide stop 22 which acts as a guide for a drive link 63, as will be described in more detail hereinbelow.

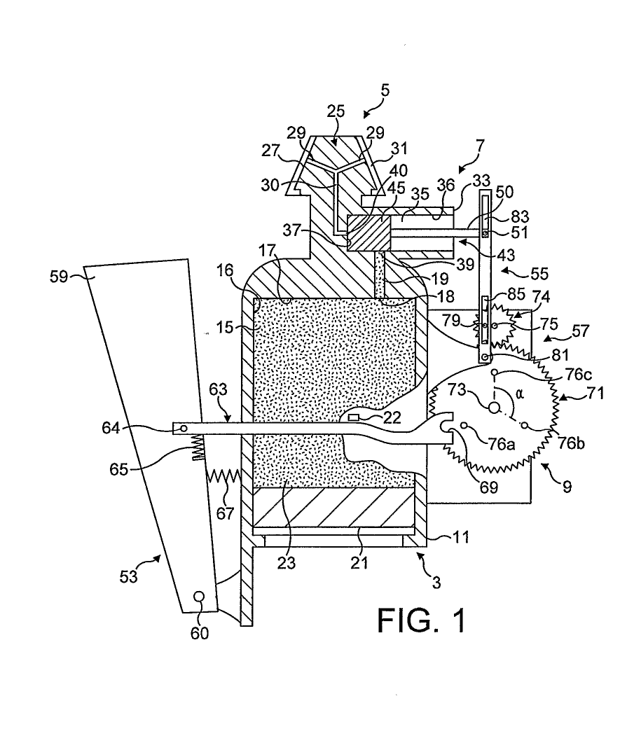

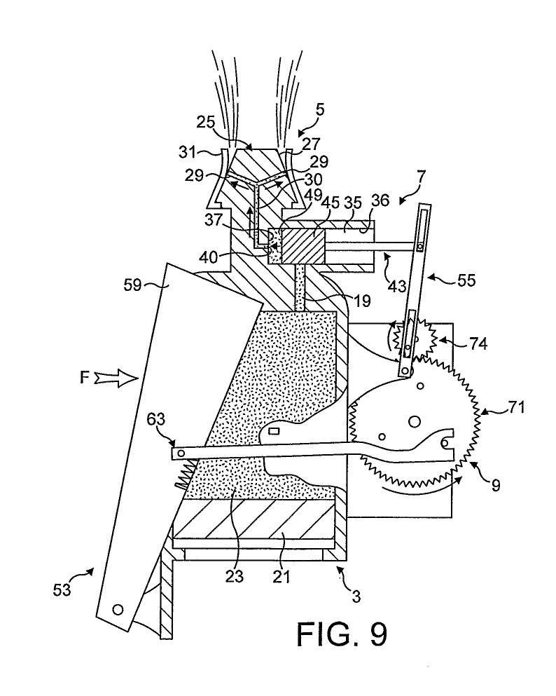

In this embodiment the outlet unit 5 is a spray nozzle which comprises a spray head 25 which includes a frusto-conical outlet surface 27 having a plurality of outlet ports 29 disposed thereabout from which substance is delivered on actuation of the delivery unit 7 and an outlet channel 30 which is fluidly connected to the outlet ports 29, and a resilient, annular skirt 31 which is disposed about the frusto-conical outlet surface 27 of the spray head 25. The skirt 31 is configured such as normally to close the delivery ports 29 from atmosphere and thereby seal the contained substance from atmosphere, and be deflected outwardly by the pressure of substance when driven through the delivery ports 29 on actuation of the delivery unit 7, with the substance being delivered as a spray from the spray head 25. FIG. 1 illustrates the skirt 31 in the normal state, where the outlet ports 29 in the spray head 25 are closed. FIG. 9 illustrates the skirt 31 in the deflected state, where substance delivered from the outlet ports 29 in the spray head 25 biases the skirt 31 outwardly and provides a spray.

The spray nozzle in this embodiment is configured as a nasal nozzle inasmuch as it is sized and shaped for insertion into a nostril of a human being.

In this embodiment the delivery unit 7 comprises a body 33 which includes a cylindrical metering cavity 35, which metering cavity 35 has a peripheral wall 36 and a closed end 37 and includes a first, transfer port 39 in the peripheral wall 36 which is fluidly connected to the transfer channel 19 in the body 11 of the storage unit 3 and a second, outlet port 40 in the closed end 37 which is fluidly connected to the outlet channel 30 in the spray head 25 of the outlet unit 5, and a piston member 43 which is slideably disposed in the metering cavity 35.

The piston member 43 comprises a piston 45 which is slideably disposed in the metering cavity 35 and a sealing fit with the peripheral wall 36 of the metering cavity 35 such as to define a metering chamber 49 ahead thereof into which substance is drawn from the reservoir 23 of the storage unit 3 on withdrawal of the piston 45 in a priming stroke, and which meters a predetermined volume of substance to the outlet unit 5 on insertion of the piston 45 in a delivery stroke, which substance, as described hereinabove, is in this embodiment delivered as a spray from the spray head 25 of the outlet unit 5.

In the priming stroke, that is, on the withdrawal of the piston 45, substance is drawn into the metering chamber 49 through the transfer channel 19 from the reservoir 23 when the piston 45 clears the transfer port 39 in the peripheral wall 36 of the metering cavity 35, with the length of the priming stroke being such as to provide that the amount of substance drawn into the metering chamber 49 exceeds that to be metered to the outlet unit 5. As will become more apparent hereinbelow, in drawing an amount of substance into the metering chamber 49 which exceeds that to be metered, the metering of the required predetermined amount of substance is ensured.

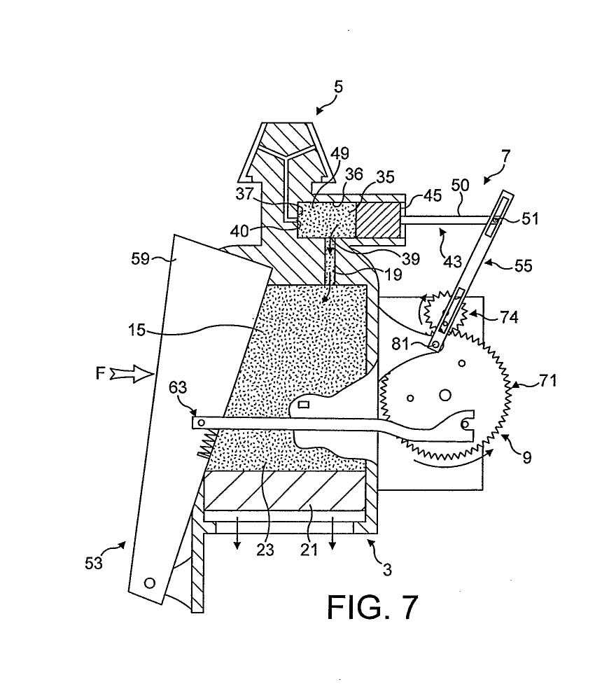

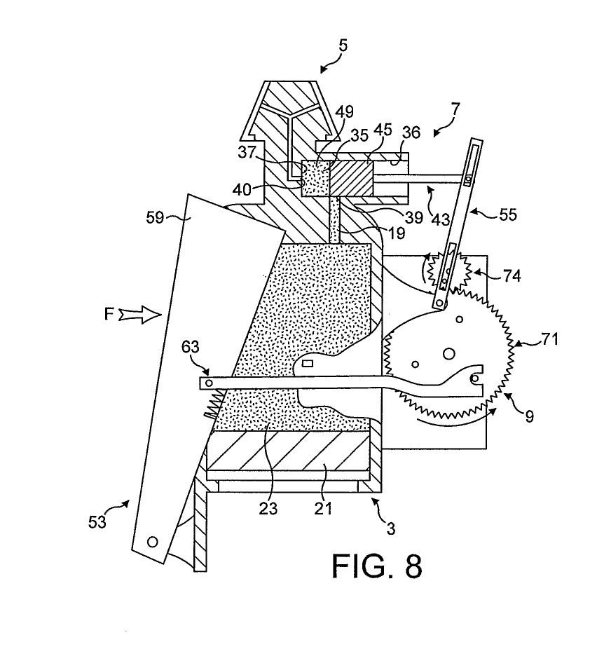

In the delivery stroke, that is, on the insertion of the piston 45 into the metering cavity 35, the piston 45 first acts to drive substance, which represents excess substance, from the metering chamber 49 through the transfer channel 19 into the reservoir 23, as illustrated in FIG. 7, with substance being driven through the transfer channel 19 in preference to the outlet channel 30 owing to the reduced flow resistance thereat. This return of excess substance to the reservoir 23 continues until the transfer port 39 in the peripheral wall 36 of the metering cavity 35 is closed by the piston 45, as illustrated in FIG. 8. Following closure of the transfer port 39, the piston 45 acts to drive the substance remaining in the metering chamber 49, which represents the required predetermined volume of substance to be metered, to the outlet unit 5. With this configuration, the axial position of the transfer port 39 in relation to the length of the delivery stroke of the piston 45 therebeyond determines the volume of substance metered by the delivery unit 7.

The piston member 43 further comprises a drive rod 50 which is connected to the piston 45 for driving the same, and the drive rod 50 includes a drive pin 51 which, as will be described in more detail hereinbelow, is engaged by a drive member 55 to drive the piston member 43.

In this embodiment the actuation mechanism 9 comprises an actuator member 53 which is manually operated by a user to actuate the delivery device, a drive member 55 which is operably coupled to the drive pin 51 on the drive rod 50 of the piston member 43 to drive the piston member 43, and a gear assembly 57 which is operably coupled to the drive member 55 such as to reciprocatingly drive the piston member 43 in the delivery and priming strokes on operation of the actuator member 53 by the user.

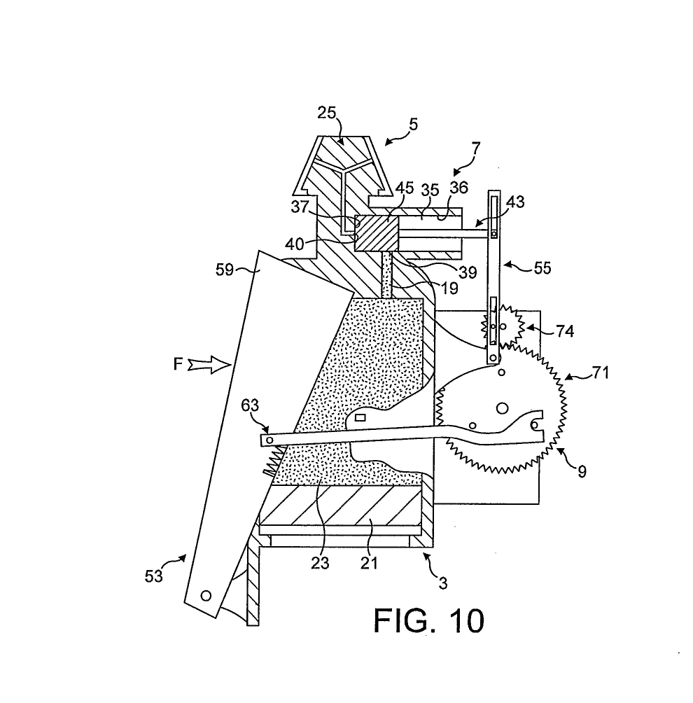

The actuator member 53 comprises an actuator button 59 which is pivoted about a pivot 60 to the body 11 of the storage unit 3 such as to be movable between a first, rest position, as illustrated in FIG. 1, which is prior to operation, and a second, actuated position, as illustrated in FIG. 10, which is following operation by the user having applied the actuation force F to the actuator button 59, a drive link 63 which is pivotally coupled about a pivot 64 to the actuator button 59 such as to be driven by the actuator button 59 on operation of the same, a first biasing element 65 which acts to bias the drive link 63 towards the guide stop 22 on the body 11 of the storage unit 3, and a second biasing element 67 which acts to bias the actuator button 59 towards the rest position such as to return the actuator button 59 to the rest position on the user releasing the actuator button 59.

In this embodiment the drive link 63 includes a recess 69 at one, the forward, end thereof for engaging one of a plurality of lugs 76 on a drive gear 71 of the gear assembly 57, as will be described in more detail hereinbelow. As will be further described in more detail hereinbelow, on depressing the actuator button 59 through the application of the actuation force F, the drive link 63, which is guided by the guide stop 22 on the body 11 of the storage unit 3, is driven forward and engages one of the lugs 76 on a drive gear 71 of the gear assembly 57.

In this embodiment the gear assembly 57 comprises a first, drive gear 71 which is mounted about a pivot 73 to the body 11 of the storage unit 3 and driven by operation of the actuator member 53, and a second, driven gear 74 which is mounted about a pivot 75 to the body 11 of the storage unit 3 and engages the drive gear 71 such as to be rotated thereby on rotation of the drive gear 71.

The drive gear 71 includes a plurality of equi-angularly arranged lugs 76 which are disposed to one face thereof and configured to be engaged by the drive link 63. In this embodiment the drive gear 71 includes three lugs 76a, 76b, 76c, each having an angular spacing a of 120 degrees about the pivot 73 thereof. With this configuration, by configuring the actuator member 53 such as to rotate the drive gear 71 through the angular spacing a between respective ones of the lugs 76a, 76b, 76c, in this embodiment 120 degrees, the drive gear 71 is rotated through the same angle, which corresponds to the angular spacing a of adjacent lugs 76a, 76b, 76c, on each operation of the actuator member 53. In other embodiments the drive gear 71 could include any number of lugs 76, where those lugs 76 are equi-angularly arranged, with the angular spacing a being 360/n degrees, where n is the number of lugs 76. For example, where the drive gear 71 includes four lugs 76, the lugs 76 would be each angularly spaced by 90 degrees.

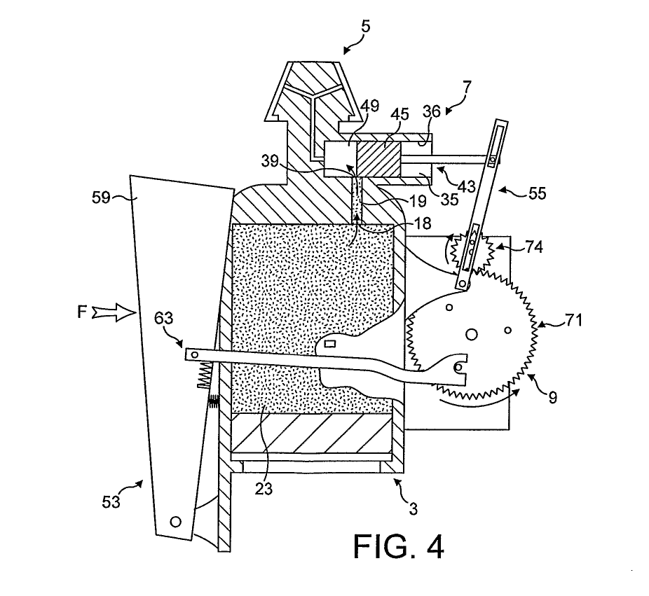

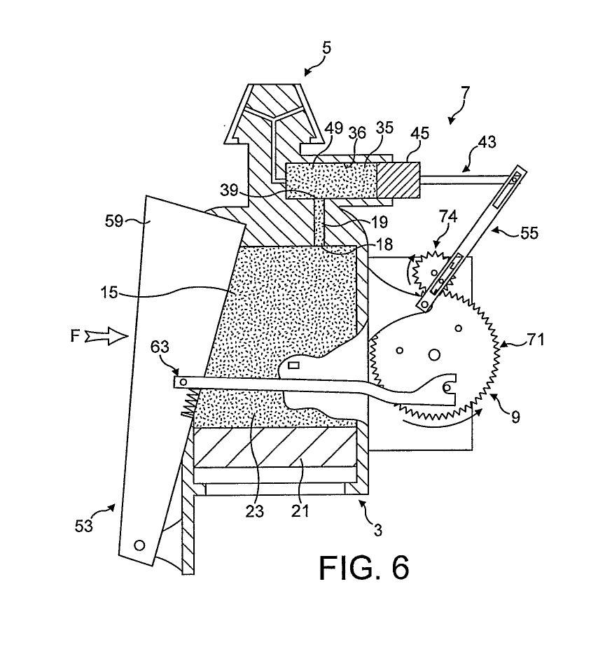

In this embodiment the driven gear 74 has a diameter which is one-third that of the diameter of the drive gear 71 such that the driven gear 74 is rotated through one complete revolution on rotation of the drive gear 71 through 120 degrees, this being the angular spacing a between adjacent lugs 76a, 76b, 76c on the drive gear 71. In other embodiments, where the drive gear 71 has n lugs 76, the driven gear 74 is sized to have a diameter which is 1/n th of the diameter of the drive gear 71. The driven gear 74 includes a single drive pin 79 which is disposed to one face thereof. The drive member 55 is pivotally coupled at one end thereof about a pivot 81, and includes a first slot 83 at the other end thereof in which the drive pin 51 on the drive rod 50 of the piston member 43 is captively disposed, and a second slot 85 at a location between the pivot 81 and the first slot 83 in which the drive pin 79 on the driven gear 74 is captively disposed. With this configuration, rotation of the driven gear 74 causes reciprocating angular rotation of the drive member 55, which in turn effects a reciprocating movement of the piston member 43. By configuring the diameter of the circle defined by rotation of the drive pin 79 on the driven gear 74, the location of the pivot 81 of the drive member 55 in relation both to the pivot 75 of the driven gear 74 and the drive pin 51 on the drive rod 50 of the drive member 43, both the combined length of the delivery and priming strokes and the relative lengths of phases of the delivery and priming strokes can be configured. In this way, the relative speeds of the delivery and priming strokes can be different, and in particular the speed of the piston 45 can be configured to be quicker in the delivery phase of the delivery stroke during which substance is delivered from the outlet port 40 in the closed end 37 of the metering cavity 35 than the priming stroke. The speed of the piston 45 is advantageously rapid in the delivery phase of the delivery stroke in order to develop the high pressure necessary to generate a spray from the outlet unit 5, and advantageously slower in the priming stroke in order to allow for the effective transfer of substance from the reservoir 23 of the storage unit 3 to the metering chamber 49 of the delivery unit 7. In this embodiment the configuration is such that the delivery phase, which corresponds to the movement of the piston 45 from the position in which the transfer aperture 39 in the peripheral wall 36 of the metering cavity 35 is closed by the piston 45, as illustrated in FIG. 8, to the fully-advanced position of the piston 45, as illustrated in FIG. 10, corresponds to 5/32 of a revolution of the driven gear 74, whereas the priming phase, which corresponds to the movement of the piston 45 from the position in which the transfer aperture 39 in the peripheral wall 36 of the metering cavity 35 is open, as illustrated in FIG. 4, to the fully-retracted position of the piston 45, as illustrated in FIG. 6, corresponds to ⅜ of a revolution of the driven gear 74, where the speed of rotation of the driven gear 74 is substantially uniform for each revolution thereof corresponding to a single actuation of the actuator button 59 of the actuator member 53.

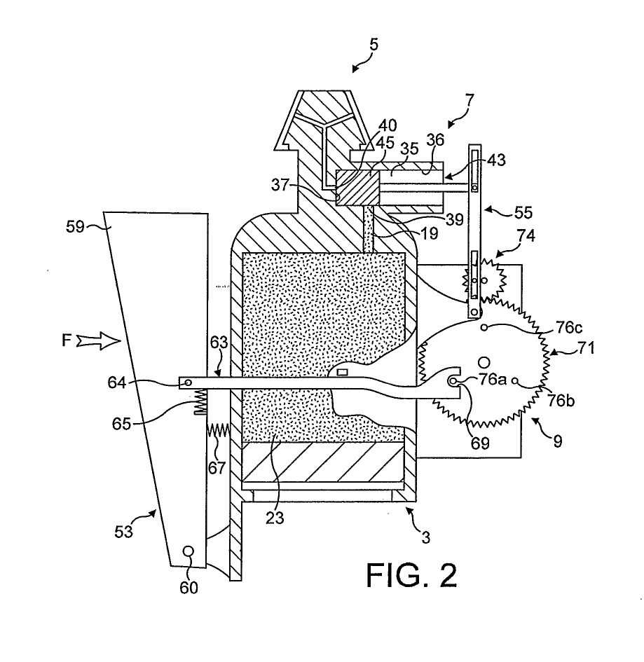

Operation of the delivery device will now be described hereinbelow with reference to FIGS. 2 to 11 of the accompanying drawings.

A user first takes the delivery device, and applies an actuation force F to the actuator button 59 of the actuator member 53. At the outset, the delivery device is in a rest configuration, in which configuration the piston 45 abuts the closed end 37 of the metering cavity 35 and closes both the transfer port 39 in the peripheral wall 36 of the metering cavity 35 and the output port 40 in the closed end 37 of the metering cavity 35, as illustrated in FIG. 2. By so configuring the delivery device in a rest configuration, the metering chamber 49 is kept sterile, enabling the potential use of preservative-free substances.

In applying an actuation force F to the actuator button 59, the actuator button 59 is pivoted about the pivot 60 thereof, with the pivoting of the actuator button 59 causing the drive link 63 to be driven forwardly into engagement with one of the lugs 76a, 76b, 76c, in this embodiment a first lug 76a, on the drive gear 71, as illustrated in FIG. 2. In being driven forwardly, the drive link 63 is guided by the guide stop 22 into engagement with the first lug 76a on the drive gear 71, and, where so engaged, the first lug 76a on the drive gear 71 is located in the recess 69 in the forward end of the drive link 63.

Following engagement of the drive link 63 with the first lug 76a on the drive gear 71, the drive gear 71 is subsequently rotated.

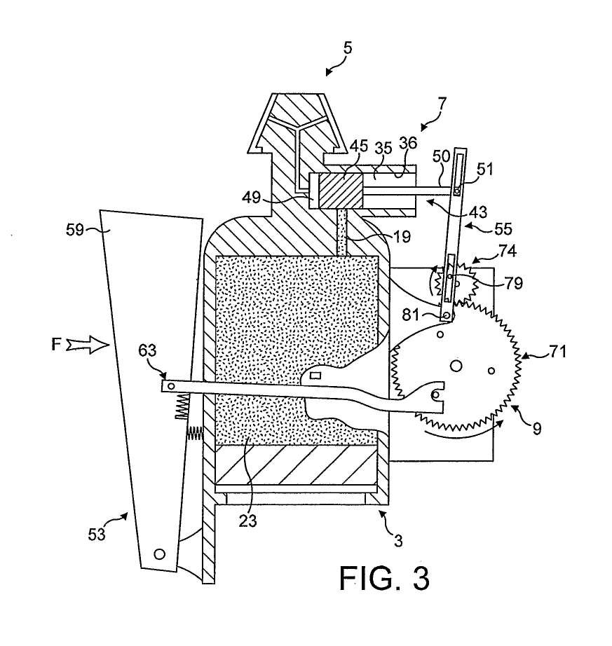

In a first phase of rotation of the drive gear 71, as illustrated in FIG. 3, the rotation of the drive gear 71 causes rotation of the driven gear 74, which rotation of the driven gear 74, through rotation of the drive pin 79 on the driven gear 74, in turn effects pivoting of the drive member 55 about the pivot 81 thereof in one sense, in this embodiment a clockwise sense as illustrated, which pivoting of the drive member 55, through engagement with the drive pin 51 on the drive rod 50 of the piston member 43, acts to withdraw the piston member 43 from the metering cavity 35 of the delivery unit 7. FIG. 3 represents the configuration following rotation of the driven gear 74 through ⅛ of a revolution.

With continued rotation of the driven gear 74, the piston member 43 is further withdrawn from the metering cavity 35 until such position, as illustrated in FIG. 4, at which the piston 45 of the piston member 43 clears the transfer port 39 in the peripheral wall 36 of the metering cavity 35, and the metering cavity 35 is in fluid communication through the transfer channel 19 with the reservoir 23 of the storage unit 3. FIG. 4 represents the configuration following rotation of the driven gear 74 through ¼ of a revolution.

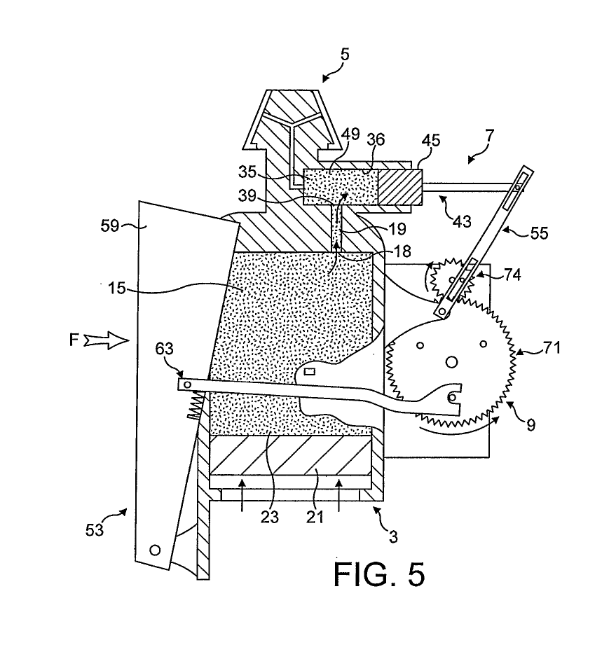

From this position, as illustrated in FIG. 5, the further withdrawal of the piston member 43 causes the piston 45 to draw substance into the metering chamber 49 through the transfer channel 19 from the reservoir 23 of the storage unit 3. As illustrated, in compensating for the withdrawal of substance from the reservoir 23, the sealing member 21 is drawn into the storage cavity 15 of the storage unit 3. FIG. 5 represents the configuration following rotation of the driven gear 74 through ½ of a revolution.

This drawing of substance into the metering chamber 49 continues until the piston member 43 reaches the end of the priming stroke, as illustrated in FIG. 6. At this position, the metering chamber 49 includes an excess of substance, which thereby ensures the delivery of the required predetermined volume of substance, as described hereinabove. FIG. 6 represents the configuration following rotation of the driven gear 74 through ⅝ of a revolution. From this it will be appreciated that the delivery stroke of the piston member 43 will be at a greater speed than the priming stroke (noting that during actuation the speed of rotation of the drive gear 71 is constant, or substantially constant).

With continued operation of the actuator member 53 through application of the actuation force F and the continued rotation of the driven gear 74, the driven gear 74 acts to pivot the drive member 55 about the pivot 81 thereof in the opposite sense, in this embodiment the counter-clockwise sense as illustrated, which pivoting of the drive member 55, through the engagement of the drive member 55 and the piston member 43 at the drive pin 51 on the drive rod 50 of the piston member 43, acts to drive the piston member 43 into the metering cavity 35 of the delivery unit 7. As illustrated in FIG. 7, which represents the configuration following rotation of the driven gear 74 through ¾ of a revolution, the forward movement of the piston member 43, and hence the piston 45 thereof, causes the piston 45 to drive substance from the metering chamber 49 through the transfer port 39 in the peripheral wall 36 of the metering cavity 35, and hence the transfer channel 19, into the reservoir 23 of the storage unit 3. As described hereinabove, substance is driven through the transfer port 39 in the peripheral wall 36 of the metering cavity 35 instead of the outlet port 40 in the closed end 37 of the metering cavity 35 owing to the lower flow resistance thereat. This flow of substance into the reservoir 23 of the storage unit 3 is accommodated by outward movement of the sealing member 21 in the storage cavity 15 of the storage unit 3.

With continued rotation of the driven gear 74 and forward movement of the piston 45 of the piston member 43, the piston 45 continues to drive substance from the metering chamber 49 through the transfer port 39 in the peripheral wall 36 of the metering cavity 35, and hence the transfer channel 19, into the reservoir 23 of the storage unit 3.

This transfer of substance continues until the piston member 43 has been moved sufficiently forwardly as to close the transfer port 39 in the peripheral wall 36 of the metering cavity 35, as illustrated in FIG. 8. FIG. 8 represents the configuration following rotation of the driven gear 74 through 27/32 of a revolution.

From this position, the further forward movement of the piston member 43 causes the piston 45 thereof to drive substance from the metering chamber 49 through the outlet port 40 in the closed end 37 of the metering cavity 35 to the outlet unit 5. At this position, the volume of substance in the metering chamber 49 represents the predetermined volume of substance to be metered by the delivery device.

As illustrated in FIG. 9, which represents the configuration following rotation of the driven gear 74 through 29/32 of a revolution, with continued forward movement of the piston member 43, the piston 45 thereof acts to drive substance from the metering chamber 49 through the outlet port 40 in the closed end 37 of the metering cavity 35 to the outlet unit 5. The substance delivered to the outlet unit 5 is delivered through the delivery channel 30 of the spray head 25 and from the delivery ports 29 in the delivery surface 27 of the spray head 25, with the substance delivered from the delivery ports 29 causing the skirt 31 of the spray head 25 to be deflected outwardly by the pressure of substance and the substance being delivered as a spray from the spray head 25.

This delivery of substance from the outlet unit 5, in this embodiment the spray head 25, continues until the actuator member 53 is fully depressed and the piston member 43 is at the end of the delivery stroke, as illustrated in FIG. 10, with the piston 45 of the piston member 43 abutting the closed end 37 of the metering cavity 35 and thereby closing both the transfer port 39 in the peripheral wall 36 of the metering cavity 35 and the outlet port 40 in the closed end 37 of the metering cavity 35. In this position, the drive gear 71 has been rotated through 120 degrees, with the lugs 76a, 76b, 76c having been rotated through 120 degrees and adopting the previous positions of adjacent ones of the lugs 76a, 76b, 76c prior to actuation of the device. FIG. 10 represents the configuration following rotation of the driven gear 74 through one revolution, and rotation of the drive gear 71 through 120 degrees, that is, ⅓ of a revolution.

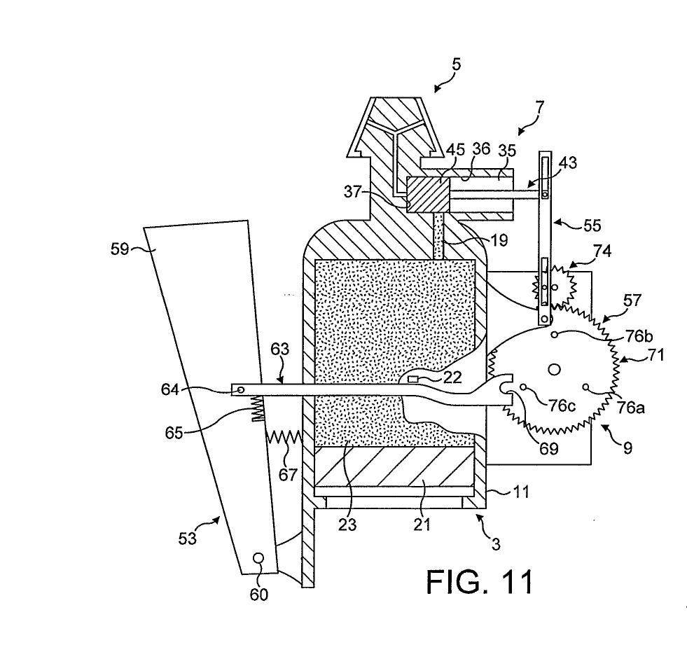

Following actuation of the device, the user then releases the actuator member 53 by removing the actuation force F from the actuator button 59 of the actuator member 53. On releasing the actuator button 59, the actuator button 59 is returned to the initial, rest position by the action of the second biasing element 67 of the actuator member 53, as illustrated in FIG. 11. The actuator button 59, in being returned to the rest position, draws the drive link 63 therewith to the operative position. The drive link 63 is biased by the first biasing element 65 of the actuator member 53 towards the guide stop 22 on the body 11 of the storage unit 3, and maintained in the operative position by the first biasing element 65 of the actuator member 53. In this position, the forward end of the drive link 63, which includes the engagement recess 69, is positioned to receive a further lug 76c on the drive gear 71 of the gear mechanism 57 on subsequent actuation of the actuation mechanism 9; this further lug 76c being that lug 76c adjacent the lug 76a engaged by the drive link 63 in the previous actuation of the device.

Finally, it will be understood that the present invention has been described in its preferred embodiments and can be modified in many different ways without departing from the scope of the invention as defined by the appended claims.

For example, in the preferred embodiment, the delivery device is configured for nasal administration, but it should be understood that the delivery device finds equal application in other administration routes, for example, oral and topical administration. For instance, the spray nozzle could be configured as an oral nozzle, e.g. a mouthpiece, instead of a nasal nozzle.

Also, the delivery device can find application in a delivery system where the delivery device delivers a metered amount of substance to another device which operates on the metered amount of substance. One such delivery system could incorporate the delivery device, where utilized simply to deliver a metered amount of substance, and conveying means to which the substance is delivered, where the conveying means conveys the substance to an outlet for delivery to an external environment. In one embodiment the conveying means can provide for a change of state of the substance. For example, the conveying means may include a vibrating element, such as a mesh, which converts a metered volume of liquid to an aerosol, typically a mist. The vibrating element could, for example, be a piezoelectric element or mesh.

Further, in the preferred embodiment, the delivery device has a medicinal application, with the substance containing a medicament, but it should be understood that the delivery device has many other non-medicinal applications, typically in the area of consumer healthcare, as in the case of toothpaste, sun cream lotion, etc.

Yet further, in the preferred embodiment, the substance is a liquid, but it should be understood that the substance can be of other kind, for example, as powders, gases, creams, pastes, etc.

Also, as regards the provision of reference signs in the appended claims, it is to be understood that reference signs are provided only for illustrative purposes and are not intended to confer any limitation to the claimed invention.

1. A delivery device for delivering a metered amount of substance on each actuation thereof, comprising:
a delivery unit operable to deliver a metered amount of substance, the delivering unit including a piston member which, in reciprocating movement, primes, meters and delivers a metered amount of substance; and
an actuation mechanism actuatable by a user to operate the delivery unit, the actuation mechanism comprising an actuator member to which a user applies an actuation force in substatially a single direction, and being configured to effect the reciprocating movement of the piston member on application of the actuation force.

2. The device of claim 1, wherein the delivery unit comprises a metering cavity in fluid communication with a reservoir for storing substance, and a piston member is reciprocatingly movably disposed in the metering cavity along a piston axis, the piston member being movable in a first direction to a first, primed position to draw substance into the metering cavity and thereby prime the delivery unit, and a second direction, opposite to the first direction, to a second, delivered position to meter and deliver a metered amount of substance from the metering cavity.

3. The device of claim 2, wherein the actuation mechanism further comprises a gear assembly which is operably coupled to the piston member and the actuator member such as to effect the reciprocating movement of the piston member on application of the actuation force to the actuator member.

4. The device of claim 3, wherein the gear assembly comprises a first, drive gear which is rotatable about a pivot and driven by actuation of the actuator member, and a second, driven gear which is rotatable about a pivot, driven by the drive gear and operably coupled to the piston member.

5. The device of claim 4, wherein the actuation mechanism further comprises a drive member which operably couples the piston member and the driven gear.

6. The device of claim 5, wherein the drive member is pivotally mounted about a pivot such as to be reciprocatingly pivoted by the driven gear.

7. The device of claim 6, wherein the drive member is configured such that the piston member is moved at a greater speed in the second direction during a delivery stroke in which substance is delivered from the metering cavity than in the first direction during a priming stroke over a length equal to the delivery stroke in which substance is drawn into the metering cavity.

8. The device of claim 7, wherein the pivot of the drive member is located in a position offset from an axis extending through the pivot of the driven gear and orthogonal to the piston axis.

9. The device of claim 6, wherein the driven gear includes a drive pin on a face thereof which engages the drive member.

10. The device of claim 3, wherein the drive gear includes n lugs on a face thereof, the lugs being equi-angularly spaced and having an angular spacing of 360/n degrees, and the actuator member is configured to rotate the drive gear through 360/n degrees on each actuation thereof.

11. The device of claim 10, wherein the driven gear has a diameter of 1/n of that of the drive gear such that, for each actuation of the actuator member, the driven gear is rotated through 360 degrees.

12. The device of claim 2, wherein the metering cavity includes a peripheral wall and the piston member includes a piston which is a sealing fit with the peripheral wall of the metering cavity, the piston having a pressure face for acting on the substance.

13. The device of claim 12, wherein the peripheral wall of the metering cavity includes a transfer port, the transfer port being located at a position between the positions of the pressure face of the piston when the piston member is in the primed and delivered positions, and providing for the transfer of substance from the metering cavity to the reservoir with movement of the piston member in the second direction until closed by the piston.

14. The device of claim 13, wherein, prior to actuation of the actuator member, the piston member is located such as to close the transfer port.

15. The device of claim 14, wherein, prior to actuation of the actuator member, the piston member is located such as to close the metering cavity from an external environment.

16. The device of claim 1, further comprising:
a storage unit including a reservoir for storing substance in fluid communication with the delivery unit.

17. The device of claim 1, further comprising:
an outlet unit in fluid communication with the delivery unit from which substance is delivered.

18. The device of claim 17, wherein the outlet unit comprises a spray nozzle.

19. A delivery device for delivering a metered amount of substance on each actuation thereof, comprising:
a delivery unit operable to deliver a metered amount of substance, wherein the delivering unit comprises a metering cavity in fluid communication with a reservoir for storing substance, and a piston member reciprocatingly movably disposed in the metering cavity along a piston axis, the piston member being movable in a first direction to a first, primed position to draw substance into the metering cavity and thereby prime the delivery unit, and a second direction, opposite to the first direction, to a second, delivered position to meter and deliver a metered amount of substance from the metering cavity; and
an actuation mechanism actuatable by a user to operate the delivery unit, the actuation mechanism comprising an actuator member to which a user applies an actuation force, and being configured to effect the reciprocating movement of the piston member on application of the actuation force, with the piston member being moved at a greater speed in the second direction during a delivery stroke in which substance is delivered from the metering cavity than in the first direction during a priming stroke over a length equal to the delivery stroke in which substance is drawn into the metering cavity.

20. The device of claim 19, wherein actuator member is configured such that a user applies the actuation force in substantially a single direction.

21. The device of claim 19, wherein the actuation mechanism further comprises a gear assembly which is operably coupled to the piston member and the actuator member such as to effect the reciprocating movement of the piston member on application of the actuation force to the actuator member.

22. The device of claim 21, wherein the gear assembly comprises a first, drive gear which is rotatable about a pivot and driven by actuation of the actuator member, and a second, driven gear which is rotatable about a pivot, driven by the drive gear and operably coupled to the piston member.

23. The device of claim 22, wherein the actuation mechanism further comprises a drive member which operably couples the piston member and the driven gear.

24. The device of claim 23, wherein the drive member is pivotally mounted about a pivot such as to be reciprocatingly pivoted by the driven gear.

25. The device of claim 24, wherein the pivot of the drive member is located in a position offset from an axis extending through the pivot of the driven gear and orthogonal to the piston axis.

26. The device of claim 23, wherein the driven gear includes a drive pin on a face thereof which engages the drive member.

27. The device of claim 22, wherein the drive gear includes n lugs on a face thereof, the lugs being equi-angularly spaced and having an angular spacing of 360/n degrees, and the actuator member is configured to rotate the drive gear through 360/n degrees on each actuation thereof.

28. The device of claim 27, wherein the driven gear has a diameter of 1/n of that of the drive gear such that, for each actuation of the actuator member, the driven gear is rotated through 360 degrees.

29. The device of claim 19, wherein the metering cavity includes a peripheral wall and the piston member includes a piston which is a sealing fit with the peripheral wall of the metering cavity, the piston having a pressure face for acting on the substance.

30. The device of claim 29, wherein the peripheral wall of the metering cavity includes a transfer port, the transfer port being located at a position between the positions of the pressure face of the piston when the piston member is in the primed and delivered positions, and providing for the transfer of substance from the metering cavity to the reservoir with movement of the piston member in the second direction until closed by the piston.

31. The device of claim 30, wherein, prior to actuation of the actuator member, the piston member is located such as to close the transfer port.

32. The device of claim 31, wherein, prior to actuation of the actuator member, the piston member is located such as to close the metering cavity from an external environment.

33. The device of claim 19, further comprising:
a storage unit including a reservoir for storing substance in fluid communication with the delivery unit.

34. The device of claim 1, further comprising:
an outlet unit in fluid communication with the delivery unit from which substance is delivered.

35. The device of claim 34, wherein the outlet unit comprises a spray nozzle.

36. (canceled)
